(19) United States
(12) Patent Application Publication
(10) Pub. No.: US 2026/0337599 A1
(43) Pub. Date: Oct. 29, 2026

(54) RAPID RECONSTRUCTION OF LARGE NUCLEIC ACIDS

(71) Applicant: MARY HITCHCOCK MEMORIAL HOSPITAL, FOR ITSELF AND ON BEHALF OF DARTMOUTH-HITCHCOCK CLINIC, Lebanon, NH (US)

(72) Inventor: Chun-Chieh LIN, Lebanon, NH (US)

(21) Appl. No.: 19/115,141

(22) PCT Filed: Oct. 2, 2023
(86) PCT No.: PCT/US2023/034301

§ 371 Date: Mar. 25, 2025

Related U.S. Application Data
(60) Provisional application No. 63/412,179, filed on Sep. 30, 2022.
(60) Provisional application No. 63/525,897, filed on Jul. 10, 2023.

(51) Int. Cl. C12N 15/10

(52) U.S. Cl. CPC C12N 15/1065

(57) ABSTRACT

A method for rapid reconstruction of nucleic acids is described. The method comprises the stochastic reconstruction of nucleic acid by simultaneous reaction of restriction enzymes and a DNA ligase. Copy number variations (CNVs) are almost ubiquitous in cancer. In many cases, somatic CNV analysis has led to the identification of oncogenic pathways and suggested molecular-defined therapeutic targets. However, current CNV analysis is laborious and time-consuming. Here, we develop iSCORED, a one-step random genomic DNA reconstruction method that enables efficient and unbiased quantitative assessment of CNV using a real-time Nanopore sequencer. By leveraging the long concatenated reads, we generate approximately 1-2 millions genomic fragments within one hour of sequencing. Analyzing a cohort of 26 brain tumors, we demonstrate 100% concordance in CNV detection when compared to clinically validated assays. In addition, concurrent tumor methylation classification is achieved without additional tissue preparation. The entire workflow is completed within a timeframe of 120-140 minutes, with an automatically generated CNV and methylation report. The ultrafast molecular analysis could be applied in the intra-operative setting and will be crucial for making informed decisions and modifying surgical plans.

### RELATED APPLICATION

This application claims priority to U.S. Provisional Application No. 63/412,179 filed on Sep. 30, 2022, and claims priority to U.S. Provisional Application No. 63/525,897 filed on Jul. 20, 2023, the contents of which are incorporated herein by reference in their entireties for all purposes.

### BACKGROUND

Molecular characterization of tumors has reshaped clinical medicine in cancer diagnosis and prognostic prediction. More importantly, the information guides therapeutic selection on a personalized basis. Many aggressive neoplasms occur locally. For instance, despite multimodal treatment, approximately 90% of glioblastoma patients recur/progress locally (median time=six months). This is partially due to therapeutic obstacles of drug penetration into lesional tissue (i.e., central nervous system). Surgery provides a unique opportunity for direct visualization and access to the tumor resection margins, making topical intraoperative molecular-based therapy an intriguing possibility. However, current molecular diagnostic platforms are laborious and time-consuming, which makes intraoperative therapy difficult to implement, and leaves systemic administration as the only option. These drawbacks add further complexities to therapeutic plans, including bioavailability, systemic side effects, and drug interactions.

Nanopore sequencing (Oxford Nanopore Technologies) is a channel-based device that allows real-time interpretation of DNA/RNA sequences (A, T, C, G, and U) by sensing the ionic current charges. With upgrades to detect epigenetic base modifications (such as 5-methylcytosine), intraoperative molecular diagnosis is recently made possible by detecting methylation profiling in brain tumors using nanopore sequencing. However, intraoperative genetic characterization remains challenging due to lengthy library preparations.

### SUMMARY

Disclosed is a method for reconstructing large nucleic acid molecules within a short time period. The method comprises simultaneously reacting nucleic acids with restriction enzymes and a DNA ligase. The disclosed method is also referred to herein as iSCORED (Irreversible Sticking Compatible Overhangs to REconstruct DNA).

In an aspect, the method for constructing a plurality of chimeric DNA molecules comprises: a) providing a source DNA molecule; and b) simultaneously reacting the source DNA molecule with: i) two or more restriction enzymes; and ii) a DNA ligase. In an embodiment, the two or more restriction enzymes have different restriction recognition sites but produce identical overhangs when cutting a DNA molecule, wherein the two or more restriction enzymes cut the source DNA molecule into smaller fragments, and the DNA ligase assembles the smaller fragments into the plurality of chimeric DNA molecules.

In an embodiment, the two or more restriction enzymes are two or more members selected from the group consisting of MseI, BfaI, CviQI, and NdeI.

In an embodiment, the two or more restriction enzymes comprise two or more members selected from the group consisting of XbaI, SpeI, AvrII and NheI.

In an embodiment, the DNA ligase is selected from the group consisting of T3 DNA ligase, T4 DNA ligase, and T7 DNA ligase.

In some embodiments, step (b) comprises two reaction processes (b1) and (b2), wherein in process (b1), the two or more restriction enzymes cut the source DNA molecule into smaller DNA fragments, and in process (b2), the DNA ligase ligates the smaller DNA fragments having complementary overhangs into a larger DNA fragment, wherein the processes (b1) and (b2) are allowed to repeat for N cycles to generate the chimeric DNA molecule, N is an integer greater than 5.

In one aspect, process (b2) may comprise two types ligations, Type I and Type II ligations: Type I ligation results from ligation of two sticky ends generated by same restriction enzyme and can be cut by the same restriction enzyme again, and Type II ligation results from ligation of two sticky ends generated by two different restriction enzymes and cannot be cut by the any of the two different restriction enzyme again.

In another aspect, the plurality of chimeric DNA molecules comprises more than 5, or more than 7, or between 5 and 15, or between 5-10, or between 7 and 8 smaller DNA fragments ligated together through Type II ligation.

In an embodiment, small fragments comprise an average length of about 80-100 bp, or about 90 bp when MseI, BfaI, CviQI, and NdeI are used.

In an embodiment, the reaction of step (b) is completed within about 30 minutes.

In an embodiment, the method may further comprise a step (c) of sequencing the plurality of chimeric DNA molecules.

In another embodiment, the chimeric DNA molecules are sequenced using nanopore sequencing.

In some embodiments, the source DNA molecule is obtained from a mammal, such as a human. In one aspect, the source DNA molecule is obtained from a tissue sample, such as a tumor sample from the mammal. In one aspect, the source DNA molecule is obtained during a surgery being performed on the mammal.

In an aspect, a chimeric DNA molecule comprising a plurality of fragments is produced by cutting a source DNA fragment with two or more restriction enzymes, and the plurality of fragments are stochastically ligated together to form the chimeric DNA molecule, wherein the two or more restriction enzymes have different restriction recognition sites but produce identical overhangs when cutting a DNA molecule. In an embodiment, said chimeric DNA molecule is not digestible by said two or more restriction enzymes.

In an embodiment, the average size of the chimeric molecule is about 1-10 kb. In another embodiment, the average size of the chimeric molecular is about 1-2 kb, or about 1-10 kb, or about 4-10 kb.

In an embodiment, an average size of the plurality of fragments is about 90 bp when MseI, BfaI, CviQI and NdeI are used.

In an embodiment, a kit for constructing a plurality of chimeric DNA molecules is disclosed. The kit may comprise i) two or more restriction enzymes; ii) a DNA ligase; and iii) an instruction to react a source DNA simultaneously with the two or more restriction enzymes and the DNA ligase to generate the plurality of chimeric DNA molecules. In one aspect, the two or more restriction enzymes have different restriction recognition sites but produce identical overhangs when cutting a DNA molecule, wherein the two or more restriction enzymes cut the source DNA molecule into smaller DNA fragments, and the DNA ligase ligates the smaller DNA fragments into the plurality of chimeric DNA molecules. In one embodiment, the two or more restriction enzymes comprise two or more members selected from the group consisting of MseI, BfaI, CviQI, and NdeI. In another embodiment, the two or more restriction enzymes comprise two or more members selected from the group consisting of XbaI, SpeI, AvrII and NheI. In another embodiment, the DNA ligase is selected from the group consisting of T3 DNA ligase, T4 DNA ligase, and T7 DNA ligase.

In an aspect, a method for reconstructing DNA for single cell genomics comprises: a) lysing single isolated cells to expose genomic DNA; and b) simultaneously reacting the genomic DNA with: i) two or more restriction enzymes, wherein the two or more restriction enzymes have different restriction recognition sites but produce identical overhangs when cutting a DNA molecule, wherein the two or more restriction enzymes cut the source DNA molecule into smaller fragments; ii) short barcodes, wherein the barcodes comprise compatible overhangs to the overhangs produced by the two or more restriction enzymes; and iii) a DNA ligase, wherein the DNA ligase assembles the smaller fragments and barcodes into the chimeric DNA molecule; c) amplifying the chimeric DNA molecule; and d) sequencing the chimeric DNA molecule. In an embodiment, the single cells are prepared by a process comprising: a) dissociating and suspending a cell population; b) diluting the cell population to form a cell solution; and c) transferring the cell solution to a container such that single cells are isolated.

### BRIEF DESCRIPTION OF THE DRAWINGS

FIG. 1 shows an integrated workflow for intraoperative histological and molecular diagnosis.

FIGS. 2A-2C depict the iSCORED reaction. FIG. 2A shows an example of iSCORED reactions using restriction enzymes generating compatible 5′ CTAG overhangs: 75% (12/16) of random ligations reach irreversible end products (black line) and 25% of them remain susceptible for further digestion (brown line). FIG. 2B shows a list of detailed ligation possibilities (shaded boxes show futile ligations). FIG. 2C shows a comprehensive analysis of digestion frequency, generated fragments, and normalized ligation efficiencies across entire human genomes. (Blue=T2T-CHM13, Brown=GRCh38).

FIGS. 3A-3D show copy number variation (CNV) detection using the iSCORED method of the present disclosure. FIGS. 3A-3B show the schematic reaction using 5′ TA cocktail restriction enzymes to create irreversible hybrid products. Sequencing the assembled products provides a structural overview of the entire genome (dashed lines=no alignment). FIG. 3C shows that reaction efficiency peaks at 30 mins. N50=weighted median of sequencing length. FIG. 3D shows that the sequenced units from 30 mins of reaction align to the GRCh38 reference genome. Zoom-in fragment distribution in EGFR gene region (green).

FIGS. 4A-4C show the target mutation detection workflow. FIG. 4A shows three hot spot mutation amplicons (black, green, and blue) are added with Type IIS sites (BsaI, recognition site in purple; cutting site in orange) for the iSCORED reaction. FIG. 4B shows randomly assembled amplicons that are sequenced and mapped to reference sequences. FIG. 4C shows the alignment of forward sequencing results (IDH1 R132 and BRAF V600) to reference.

FIG. 5 shows the proposed workflow of single-cell CNV with iSCORED

FIG. 6 shows the iSCORED method for ultrafast copy number variation analysis. (a) iSCORED schematic showing simultaneous compatible end ligation with TA enzyme cocktail (T\hat{ }TAA, C\hat{ }TAG, G\hat{ }TAC and CA\hat{ }TATG by MseI, BfaI, CviQI and NdeI, respectively). (b) Long stochastically concatenated DNA molecules are analyzed with Nanopore device and aligned to the reference for genome-wide quantitative measurement. (c) The reconstruction efficiency of four iSCORED cocktail combinations are compared. The reaction is incubated at 37 °C for 30 mins. CATG cocktail: NcoI (C\hat{ }CATGG), PciI (A\hat{ }CATGT), BspHI (T\hat{ }CATGA). CTAG cocktail: NheI (G\hat{ }CTAGC), SpeI (A\hat{ }CTAGT), AvrII (C\hat{ }CTAGG), XbaI (T\hat{ }CTAGA). CG cocktail: MspI (C\hat{ }CGG), HinPII (G\hat{ }CGC), HpyCH4IV (A\hat{ }CGT) and TaqI-V2 (T\hat{ }CGA). EcoRV is employed as a control since it generates blunt ends upon restriction digestion. (d) Optimization of iSCORED reaction by adjusting various experimental parameters, such as incubation periods, DNA ligases and intermittent mixing and cooling. (e) An oligodendroglioma sample was processed either sequentially (digestion, purification, and ligation), with iSCORED, or sequenced as native gDNA. Samples were normalized to contain the same amount of sequencing data. The number of unique fragments mapped per genomic bin are shown for each sample (left panels). The resulting CNV plots are shown in the right panels (resolution=600 kb per bin). CoV for sequential approach, iSCORED and native gDNA sequencing are 0.57, 0.54 and 3.3, respectively. (f) Comparison of library preparation times across three methods. The sequential DNA digestion and ligation required 150 minutes, while the iSCORED required 75 minutes and the native DNA method required 45 minutes. The goal of intraoperative molecular diagnosis is achieved within 120-150 minutes of receiving the resected specimen.

FIG. 7 shows comprehensive analysis of candidate REs for iSCORED. (a) List of all enzymatic combinations for iSCORED (4 nt overhang in left panel and 2 nt overhang in right panel). The numbers denote available REs to produce such overhangs and numbers in the parentheses denote REs sensitive to CpG methylation. (b) In silico estimation of NTAN cocktail mix using the complete human genome database as a reference (T2T-CHM13). (c) Quantitative electrophoresis (Agilent) to assess the digestion efficiencies of restriction endonuclease generating TA overhang and iSCORED reaction.

FIG. 8 shows distribution of concatenation numbers and sequenced lengths in iSCORED reactions utilizing cocktail combinations generating 2-nt and 4-nt 5′ overhangs.

FIG. 9 shows normalization of variable mapped fragments in predefined bins for accurate copy number detection. (a) The number of mapped fragments per bin fluctuates across the wild-type genome (intrinsic regional variability, IRV), yielding a relatively high coefficient of variation (CoV) of 0.68 and hampering detection of true outliers. (b) Extensive sequencing does not address the fluctuation due to IRV (left panel). Normalizing the samples with the control wild-type dataset, the CoV dramatically drops and stabilizes at ~1 million mapped fragments (right panel). (c) The control genome data displayed CoV of 0.09 after normalization (upper panel). Application of this approach allows for detecting regions of amplification in both chromosome 2 and chromosome 19 (defined as ratio≥5). (d) Mixture of tumor with wild-type gDNAs shows that the amplified copies increase as the tumor percentage increases. Using Z values of 10 as cutoff, the genetic amplification CCNE1 in chromosome 19 could be reliably detected at 5% tumor purity with 500,000 mapped fragments.

FIG. 10 shows the correlation of regional variability between wildtype datasets. The X-axis in all panels represents the proportion of NA12878, which is used for normalization in all samples. The color in the scatter plots reflects the kernel density map where the blue and red data-points correspond to low and high density, respectively.

FIG. 11 shows minimum thresholds and mapped fragments to exclude false positive hits. (a) False positive hits were eliminated from the full datasets of control gDNA samples by setting a minimum threshold of 0.2 percentile. (b) The mapped fragments count of 200k or higher, in conjunction with the 0.2 percentile threshold is sufficient to eliminate all false positive hits.

FIG. 12 shows the method of determining the detection threshold for regions with low amplification. Focal gene amplification (11×) was detected in chromosome 2 of an adenosquamous carcinoma.

FIG. 13 shows the iSCORED pipeline which allows simultaneous methylation classification of primary CNS tumors. (a) Minimal methylation classification features are acquired within 45 minutes of MinION sequencing. (b) Calibrated methylation classification scores for glioblastoma, medulloblastoma and oligodendrogliomas with high tumor purity were calculated across multiple time points from the initiation of sequencing. (c) Correct methylation classification of CNS tumors depends on high tumor purity. (d) In silico mixture of glioblastoma, medulloblastoma and oligodendrogliomas with control brain tissue dataset at various ratios (total data quantity after one-hour of sequencing).

FIG. 14 shows methylation classification of primary CNS tumors. (a) Calibrated scores across various intervals from the initiation of sequencing. (b) Calibrated scores of the six correctly classified glioblastomas and corresponding tumor percentages by histological estimation. (c) Comparison of methylation classification with intact gDNA (non-iSCORED) and iSCORED datasets with three oligodendroglioma cases.

FIG. 15 shows workflow of iSCORED for ultrafast molecular diagnosis.

FIG. 16 shows assessment of reused MinION flowcells. a) Total sequenced data (Mb) and mapped fragments from MinION and flongle runs. The usable MinION flowcells generate (1.38±0.08)×10⁶ mapped fragments within one hour whereas flongles generate (2.9±0.23)×10⁶ mapped fragments during 24 hours of sequencing. b) Exact sequencing data and mapped fragments of all samples in the reused MinION flowcells.

FIG. 17 shows the workflow of our intraoperative analysis pipeline. Before beginning to sequence a configuration file is modified to the appropriate run parameters. Once sequencing begins the analysis pipeline is initiated. It periodically gathers the new fast5 files, basecalls them, extracting modification information, filters them and aligns them with the appropriate parameters for methylation and CNV/amplification analysis. At 40 minutes the aligned files for methylation are merged, the methylation call pileup is produced and used to classify the methylation class of the sample. At 55 minutes the aligned files for the CNV/amplification analysis are merged, one read from each duplex pair is removed, and the fragments per bin are calculated and used in the generation of a CNV plot and amplification analysis.

FIG. 18 shows EGFR amplification of glioblastomas in current study.

FIG. 19 shows Table 1 showing comparison of CNV results between iSCORED and clinically validated assays.

FIG. 20 shows Table 2 showing comparison of CNV assays with iSCORED.

### DETAILED DESCRIPTION

The present disclosure relates to a method (also referred to herein as iSCORED) of reconstructing large nucleic acid molecules by employing simultaneous restriction endonuclease and DNA ligase reactions in vitro. The reconstructed nucleic acids generated by the methods of the present disclosure may be sequenced following the reactions.

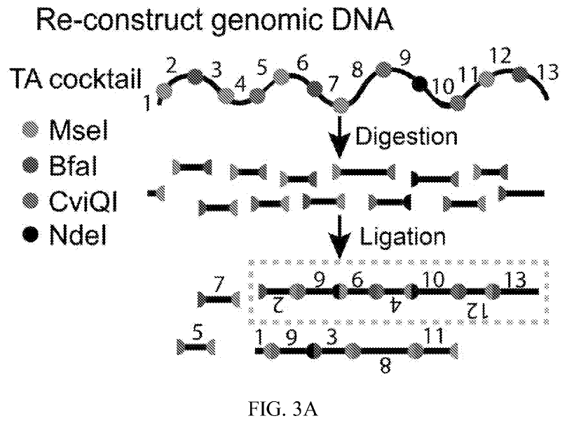

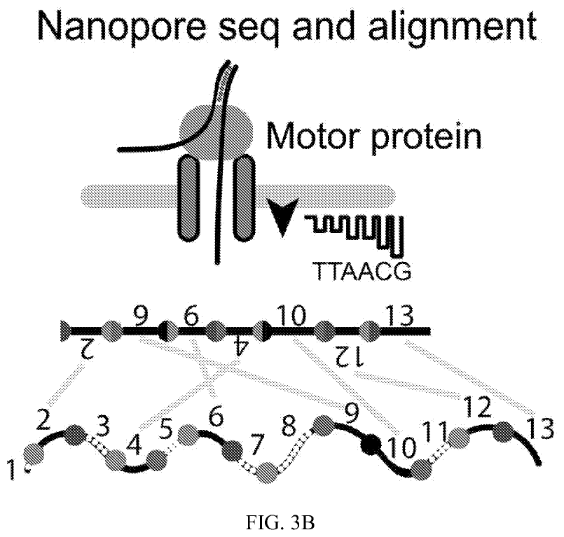

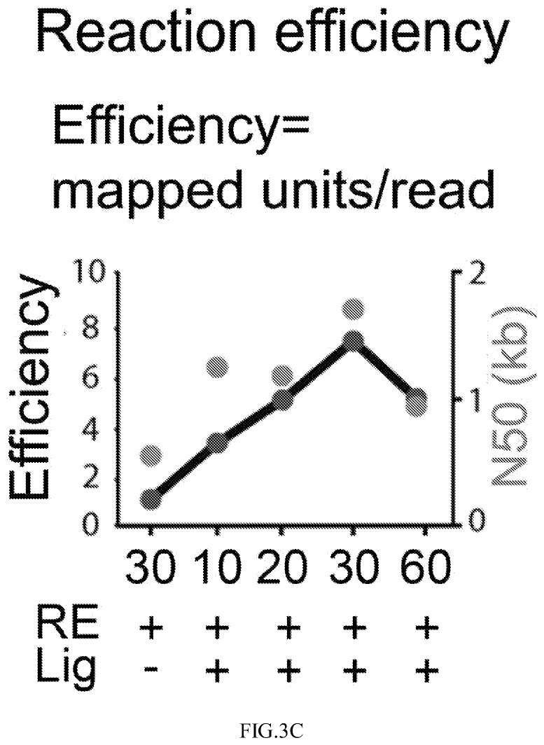

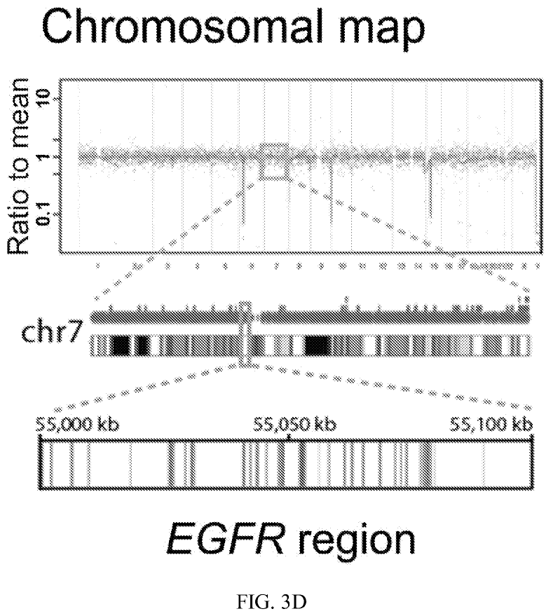

The present disclosure comprises a method for rapid reconstruction of large nucleic acids by simultaneously employing restriction endonucleases and a DNA ligase in an in vitro reaction. A mixture of restriction enzymes capable of generating compatible overhangs are utilized to fragment large nucleic acid molecules into small fragments (units). Each unit exhibits compatible cohesive ends that are amenable to random ligation with other counterparts. Within the same reaction, DNA ligase catalyzes random re-ligation of all digested units. When the cohesive ends are produced from different restriction enzymes, the “hybrid” ligation produces a stable irreversible result (FIG. 3A). This unidirectional reaction is possible because of the staggered nature of DNA recognition sequence and actual phosphodiester bond breakage. As a non-limiting example, XbaI and SpeI recognize TCTAGA and ACTAGT, respectively. Upon enzymatic digestion, both release cohesive 5′CTAG overhangs, which could re-ligate to TCTAGA, ACTAGT, TCTAGT, and ACTAGA. While the first two products are susceptible to further digestion (by XbaI and SpeI, respectively, brown line in FIG. 3A), TCTAGT and ACTAGA are the final end products. The likelihood of forming such irreversible ligations increases with the number of different restriction enzymes with compatible ends (FIG. 3B).

The methods described herein along with subsequent Nanopore sequencing allows for the capability to detect copy number variations (CNVs) and molecular mutations that are (1) diagnostically essential and/or (2) therapeutically targetable.

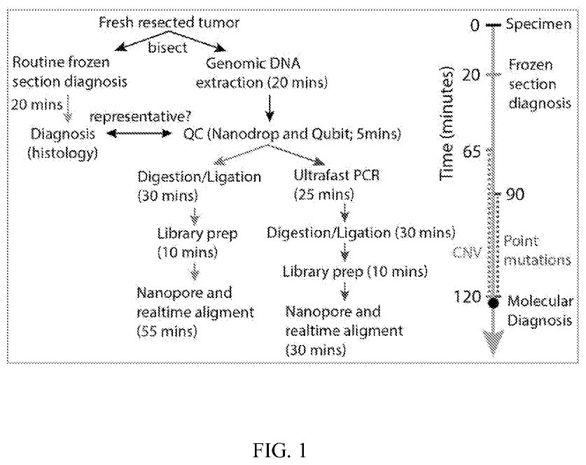

In another embodiment, the use of Nanopore sequencing with the method of the present disclosure helps identify key molecular alterations of neoplasms intraoperatively (in less than 120 minutes). FIG. 1 shows the integrated workflow for histological and molecular diagnosis. Fresh tissue acquired from the operation room is bisected for histological and molecular analysis. The result of conventional frozen section diagnosis provides valuable quality assurance of analyzed genomic DNA (green arrow). Molecularly, genomic DNA is extracted (20 mins; QIAmp DNA Micro Kit), followed by quality (Nanodrop, Thermo Fisher) and quantity (Qubit, Thermo Fisher) assessment. Approximately 300-500 ng of high-quality DNA (OD260/280=1.8−2.0) is reconstructed with iSCORED method, followed by sequencing adaptor ligation—and sequencing (55 mins). In parallel, selected diagnostic and targetable mutations are amplified by ultra-fast PCR (<25 mins, blue arrow), followed by iSCORED reaction (30 mins) and library preparation (10 mins). Generated data is analyzed in real-time to render a molecular diagnosis. The entire molecular diagnostics workflow takes 120 mins. The simplicity and time-effectiveness of this reaction allows for rapid library preparation, which makes it possible for intraoperative molecular diagnosis. The randomly assembled DNA fragments acquire its uniqueness and could be used as unique molecular identifiers (intrinsic barcodes) to trace its original templates, circumventing the long-standing PCR amplification bias problem in single cell whole genome amplification.

The iSCORED circumvents sequential experimental steps for stochastic DNA construction (i.e. DNA fragmentation by mechanical shearing and/or enzymatic digestion, followed by purification, and ligation). Sequential experimental steps (mechanical shearing and/or enzymatic digestion, purification and ligation) could reach similar DNA reconstruction results; however, it is not possible to accomplish this within short periods of time (120-150 mins in the intraoperative molecular diagnosis application). Also, the multistep procedures prohibit its application to the cellular level given minute amount of genomic DNA in single cells (5-10 pico-grams).

The instant disclosure may be further illustrated by the following items:

Item 1: A method for constructing a plurality of chimeric DNA molecules, comprising:
a) providing a source DNA molecule; and
b) simultaneously reacting the source DNA molecule with: i) two or more restriction enzymes; and ii) a DNA ligase to obtain the plurality of chimeric DNA molecules, wherein the two or more restriction enzymes have different restriction recognition sites but produce identical overhangs when cutting a DNA molecule, the two or more restriction enzymes cut the source DNA molecule into smaller DNA fragments, and the DNA ligase ligates the smaller DNA fragments into the plurality of chimeric DNA molecules.

Item 2: The method of Item 1, wherein the two or more restriction enzymes comprise two or more members selected from the group consisting of MseI, BfaI, CviQI, and NdeI.

Item 3: The method of Item 1, wherein the two or more restriction enzymes comprise two or more members selected from the group consisting of XbaI, SpeI, AvrII and NheI.

Item 4: The method of any preceding Items, wherein the DNA ligase is selected from the group consisting of T3 DNA ligase, T4 DNA ligase, and T7 DNA ligase.

Item 5: The method of any preceding Items, wherein step (b) comprises two reaction processes (b1) and (b2), wherein in process (b1), the two or more restriction enzymes cut the source DNA molecule into smaller DNA fragments, and in process (b2), the DNA ligase ligates the smaller DNA fragments having complementary overhangs into a larger DNA fragment, wherein the processes (b1) and (b2) are allowed to repeat for N cycles to generate the chimeric DNA molecule, N is an integer greater than 5.

Item 6: The method of any preceding Items, wherein process (b2) comprises two types of ligations, Type I and Type II ligations, wherein type I ligation results from ligation of two sticky ends generated by same restriction enzyme and can be cut by the same restriction enzyme again, and Type II ligation results from ligation of two sticky ends generated by two different restriction enzymes and cannot be cut by the any of the two different restriction enzyme again.

Item 7: The method of any preceding Items, wherein the plurality of chimeric DNA molecules comprises more than 5 smaller DNA fragments ligated together through Type II ligation.

Item 8: The method of any preceding Items, wherein the smaller DNA fragments have an average length of about 80-100 bp, or about 90 bp (base pairs) when using MseI, BfaI, CviQI and NdeI.

Item 9: The method of any preceding Items, wherein the reaction of step (b) is completed within 30 minutes or less.

Item 10: The method of any preceding Items, further comprising a step (c) of sequencing the plurality of chimeric DNA molecules.

Item 11: The method of any preceding Items, wherein the sequencing is performed by using nanopore sequencing.

Item 12: The method of any preceding Items, wherein result from the sequencing is analyzed to obtain information selected from the group consisting of copy number variation (CNV), methylation classification, and combination thereof.

Item 13: The method of any preceding Items, wherein the source DNA molecule is obtained from a mammal.

Item 14: The method of any preceding Items, wherein the source DNA molecule is obtained from a tumor sample of the mammal.

Item 15: The method of any preceding Items, wherein the source DNA molecule is obtained during a surgery being performed on the mammal.

Item 16: The method of any preceding Items, wherein steps (a)-(c) are performed before the surgery is completed and results from steps (a)-(c) are used to guide a surgeon performing the surgery or for implementation of molecularly targeted therapies.

Item 17: A chimeric DNA molecule comprising a plurality of DNA fragments produced by cutting a source DNA fragment with two or more restriction enzymes, wherein the plurality of fragments is stochastically ligated together to form the chimeric DNA molecule, and wherein the two or more restriction enzymes have different restriction recognition sites but produce identical overhangs when cutting a DNA molecule, wherein said chimeric DNA molecule are not digestible by said two or more restriction enzymes.

Item 18: The chimeric DNA molecule of Item 17, wherein the chimeric DNA molecule comprises more than 5 of the plurality of DNA fragments.

Item 19: The chimeric DNA molecule of any of Items 17-18, wherein an average length of the plurality of fragments is between 80-100 bp when using MseI, BfaI, CviQI and NdeI.

Item 20: The chimeric DNA molecule of any of Items 17-19, wherein an average length of the plurality of fragments is 90 bp when using MseI, BfaI, CviQI and NdeI.

Item 21: A method for reconstructing DNA for single-cell genomic analysis, comprising
a) lysing single isolated cells to expose genomic DNA in the cells;
b) simultaneously reacting the genomic DNA with: i) two or more restriction enzymes, wherein the two or more restriction enzymes have different restriction recognition sites but produce identical overhangs when cutting a DNA molecule, wherein the two or more restriction enzymes cut the source DNA molecule into smaller DNA fragments; ii) short barcodes, wherein the barcodes are unique DNA sequences comprising compatible overhangs to the overhangs produced by the two or more restriction enzymes; and iii) a DNA ligase, wherein the DNA ligase ligates the smaller DNA fragments and barcodes into a chimeric DNA molecule;
c) amplifying the chimeric DNA molecule; and
d) sequencing the chimeric DNA molecule.

Item 22: The method of Item 21 wherein the single cells are prepared by a process comprising a) dissociating and suspending a cell population; b) diluting the cell population to form a cell solution, and c) transferring the cell solution to a container where single cells are isolated.

Item 23: A kit for constructing a plurality of chimeric DNA molecules, comprising: i) two or more restriction enzymes; ii) a DNA ligase; and iii) an instruction to react a source DNA simultaneously with the two or more restriction enzymes and the DNA ligase to generate the plurality of chimeric DNA molecules; wherein the two or more restriction enzymes have different restriction recognition sites but produce identical overhangs when cutting a DNA molecule, wherein the two or more restriction enzymes cut the source DNA molecule into smaller DNA fragments, and the DNA ligase ligates the smaller DNA fragments into the plurality of chimeric DNA molecules.

Item 24: The kit of Item 23, wherein the two or more restriction enzymes comprise two or more members selected from the group consisting of MseI, BfaI, CviQI, and NdeI.

Item 25: The method of Item 23, wherein the two or more restriction enzymes comprise two or more members selected from the group consisting of XbaI, SpeI, AvrII and NheI.

Item 26: The method of any of Items 23-25, wherein the DNA ligase is selected from the group consisting of T3 DNA ligase, T4 DNA ligase, and T7 DNA ligase.

It will be readily apparent to those skilled in the art that the methods described herein may be modified and substitutions may be made using suitable equivalents without departing from the scope of the embodiments disclosed herein. Having now described certain embodiments in detail, the same will be more clearly understood by reference to the following examples, which are included for purposes of illustration only and are not intended to be limiting.

### Examples

### Example 1: Copy Number Variation Analysis of Tumor Genome

Sequencing randomly assembled genomic molecules provides insight into chromosomal integrity at a genome-wide level. This allows “sampling” the genome without actual sequencing its entirety. The concept was proposed and tested; however, the lengthy protocols make it incompatible with the intraoperative application. The iSCORED method of the present disclosure could fragment the genome into short DNA units and concatenate them into long molecules within 30 minutes.

To maximize random sampling without overburdening the sequencing capacity, 4-mer and 6-mer Type IIP restriction enzymes were selected to digest genomic DNA into the smallest sufficient units for unique genomic mapping (FIGS. 3A-3B). Specifically, restriction enzymes producing 5′TA overhangs are used: MseI, BfaI, CviQI and NdeI collectively generate DNA fragments with a mean length of 90.03 bp (total cleavage sites of 34,624,810 with CHM13 reference genome). The DNA reconstruction efficiency peaks at around 20 to 30 mins, at which one read consists of up to 10 unites (FIG. 3C). MinION flowcells (R9.4.1) generate approximately 100,000-150,000 reads/hour (length<5,000 bp; ONT). Normalizing to 100,000 reads (1 hour of sequencing), this platform generates an average genomic resolution of 3.3 kb covering 5.4% of the entire genome (FIG. 3D). An even higher resolution could be reached by continuing sequencing. This result is superior to current SNP-based Affymetrix OncoScan, which has an average genomic resolution of 9.6 kb and takes 2-3 days.

### Example 2: Point Mutations of Diagnostically and Therapeutically Important Targets

A panel of diagnostically important and/or therapeutically targetable mutations was selected (Table 1a, bold=available molecularly targeted drugs; number=hot spot mutations sites). Published NGS primer sets are used to increase the success rate (Thermo Fisher #4475346). First, an ultra-fast thermocycler (UF-100, Victory Scientific) with a high ramping rate (8.0 °C/sec) is used to amplify uniformly short PCR fragments (150-400 bp), aiming to minimize reaction time (<25 mins) and incorporate them into multiplex PCR. Second, the mixed amplicons are concatenated into long DNA fragments with the digestion/ligation protocol. This increases the coverage of individual genes with fewer required adaptors, taking full advantage of the long-read capacity of Nanopore sequencing.

TABLE 1a
Mutation targets

| | |
|---|---|
| BRAFV600 (1) | H3F3A (1) |
| CTNNB1 (1) | IDH1 R132 (1) |
| EGFR (8) | IDH2 R172 (1) |
| FGFR1 (2) | PIK3CA (4) |
| FGFR2 (4) | PDGFRA (4) |
| FGFR3 (5) | SMARCB1 (4) |
| HIST1H3B (2) | TERT promoter (1) |

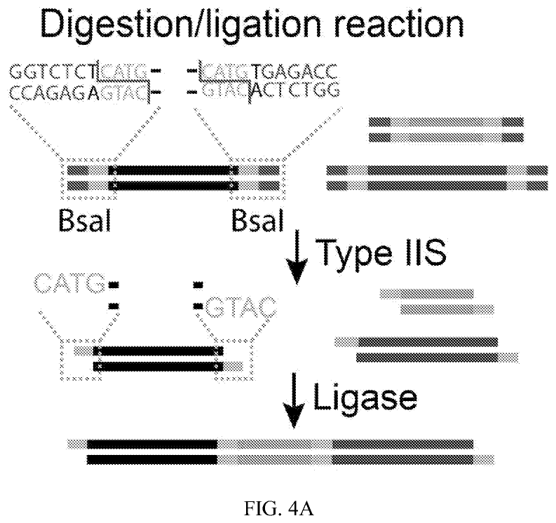

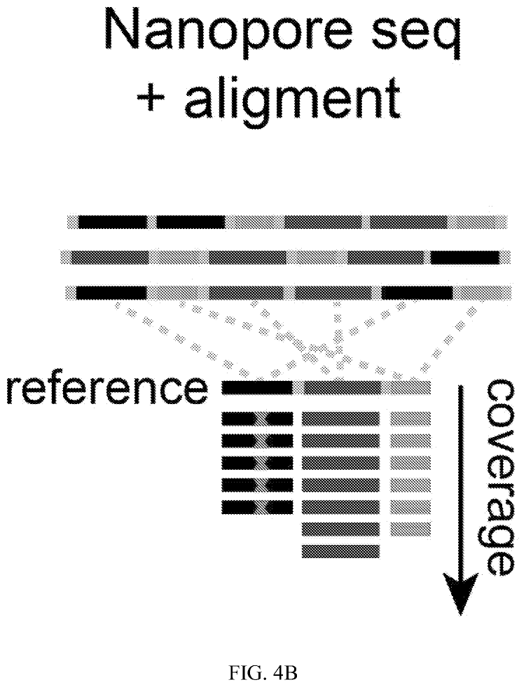

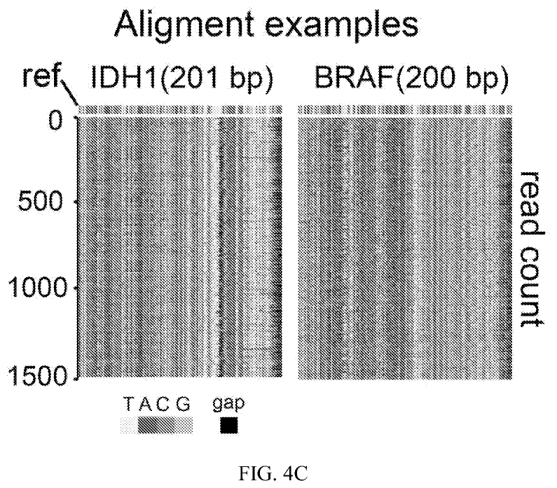

To optimize irreversible reactions, the amplified DNAs are incorporated with Type IIS restriction enzyme sites during PCR reaction (i.e., BsaI; FIG. 4A). As opposed to conventional Type IIP restriction enzymes used for CNV analysis above, Type IIS restriction enzymes recognize asymmetric DNA sequences and cut outside of recognition sequences at a defined distance (NEB). Thus, the overhangs are not constrained by the enzyme recognition sequence, allowing completely irreversible ligation of digested PCR products. Unique overhangs are also used for complex cloning in the synthetic biology field (i.e., Golden gate cloning).

Here, each PCR product was purposefully designed to expose a 5′ CATG overhang after Type IIS enzyme digestion—each amplicon loses Type IIS recognition sites and gains the ligation ability (FIG. 4A and FIG. 4B). All single hot spot mutation genes were successfully amplified within 25 mins (compared to 45 mins by C1000 Bio-Rad Thermal Cycler with the same protocol). The digestion/ligation reaction reaches peak ligation efficiency at 30 mins by electrophoresis analysis. Preliminary alignment results showed >3,000× coverage of sequence 25-30 potential genetic mutations at 1,000× coverage within 120 mins upon receipt of the specimen.

### Example 3: iSCORED for Single-Cell Genomics

iSCORED may be applied to single-cell genomics for unbiased quantification of genetic elements. To achieve this, two molecular barcode systems are employed: first, short internal barcodes with compatible 5′ overhangs are used in the single-cell iSCORED reaction to uniquely identify reads from the same cell. The process is compatible with the existing ligation reaction in iSCORED and will allow demultiplexing of sequencing reads for cellular origins. Second, the intrinsic barcode information is naturally embedded in the stochasticity of DNA reconstruction. Each chimeric DNA molecule after the iSCORED is essentially unique, and the feature will be used to deconvolute sequenced amplicons to the unique original templates. Thus, the genetic quantitative uncertainty due to amplification bias is circumvented. For instance, if gene X is duplicated, during stochastic DNA reconstruction, the two copies of gene X are ligated with different partners and form unique chimeric DNA molecules.

The traceable information (intrinsic barcodes) is used to compile the same sequenced amplicons to generate original chimeric templates (deconvolution). Therefore, the necessary DNA amplification only increases the sequencing depth and accuracy of the chimeric molecules but does not create quantitative bias for the CNV analysis.

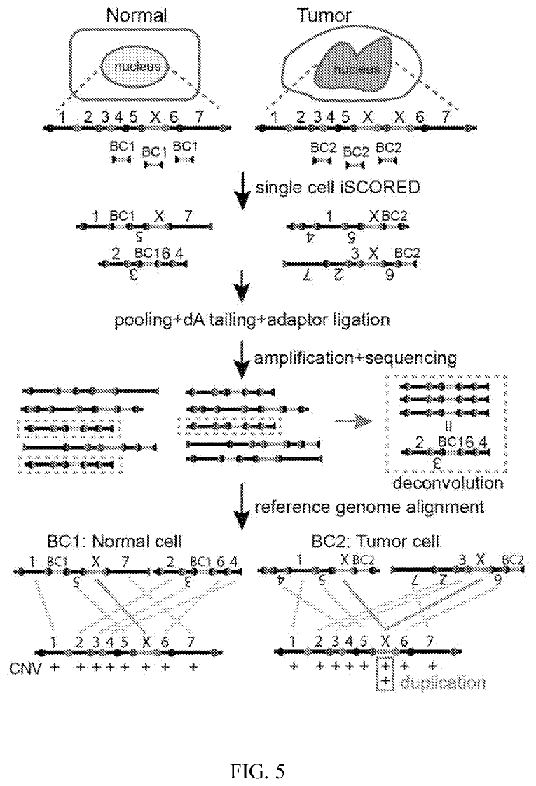

Several steps are employed to adapt iSCORED for single-cell genomics (FIG. 5). First, homogenous cell populations are dissociated and suspended, such as fresh human buccal mucosal cells and frozen glioblastoma cells. The suspended cells are serially diluted and manually pipetted to 96 well plates. Each well contains 2.5-μl lysis buffer, and individually isolated cells will be lysed at 50 °C for 1 hour and 70 °C for 15 minutes. After that, 7.5-μl of iSCORED solution with internal barcode mixtures is added for stochastic DNA reconstruction and individual cell barcoding. Second, the barcoded chimeric DNA molecules are pooled and purified with phenol-chloroform extraction. End repair and dA-tailing are completed to enable ligation to adaptors with a single T overhang. Third, established Illumina NGS reagents are used for library amplification. Briefly, the NEBNext hairpin adaptors are ligated to the pooled dA-tailed chimeric DNA molecules, followed by USER® enzyme to remove uracils and PCR enrichment. The results are analyzed with quantitative electrophoresis to assess amplicon size distribution. To seamlessly ligate to Nanopore sequencing adaptors, Deep Vent (exo-) DNA polymerase (NEB) is used, which exhibits dA-tailing property and 5× higher fidelity than Taq polymerase. Finally, the amplicon library is purified and ligated to sequencing adaptors for Nanopore sequencing. The sequencing reads are deconvoluted to original chimeric templates and aligned to the reference genome (FIG. 5).

### Example 4 Nanopore-Based Random Genomic Sampling for Intraoperative Molecular Diagnosis

In order to determine whether the instant methodology would work in a clinical setting, nanopore-based random genomic sampling was performed for intraoperative molecular diagnosis.

Copy number variations (CNVs) contribute to cancer development and progression by activating oncogenes and inactivating tumor suppressor genes¹⁻³. As a predominant class of genomic alterations, CNVs are also involved in a wide range of biological processes, including human evolution⁴⁻⁶, neurodegeneration^{7,8}, and developmental disorders^{4,9-12} Despite the importance of CNVs in genomic biology, characterizing CNVs remains laborious and time-consuming. Current approaches rely on nucleotide hybridization^{5-7,10,11,13-15} and next-generation sequencing^{1-3,8}, with a turnaround time of several days to weeks which could delay clinical therapeutic plans^{16,17}. In contrast, Nanopore sequencing (Oxford Nanopore Technologies, ONT) is a channel-based device that provides real-time interpretation of long-read nucleotide sequences. Despite some success in application of Nanopore sequencing in ultrafast CNV diagnostics^{18,19} the genomic resolution is limited to 10 Mb due to low numbers of aligned DNA fragments within a short sequencing timeframe¹⁹

Ultrafast high-resolution CNV detection can be achieved by analyzing randomly concatenated DNA fragments. The approach enables the identification of multiple mappable DNA fragments in one sequencing read, thus optimizing sequencing efficacy. By sequencing a fraction of randomly assembled genomic fragments, the genome-wide chromosomal integrity can be quantitatively assessed. While previous attempts involving quantitative genomic analysis have been introduced, the methods are lengthy and require sequential mechanical shearing and/or enzymatic digestion, purification, and ligation^{20,21}. An important rate-limiting factor has been the lack of a highly efficient method to reliably process genomic DNA in one reaction.

Here, we introduce iSCORED, irreversible Sticking Compatible Overhang to Reconstruct DNA, a method to stochastically reconstruct genomic DNA by a simultaneous digestion and ligation reaction. The technology features a simple and time-effective sequencing library preparation that allows ultrafast assessment of genome-wide copy number variations in the intraoperative setting (2-2.5 hours after receiving specimen). Furthermore, by leveraging the epigenetic modifications available through Nanopore sequencing, we demonstrated the feasibility of concurrent methylation classification in primary CNS tumors. The pipeline was applied to a cohort of 26 intracranial neoplasms, consisting of 17 primary CNS tumors and 9 metastatic tumors. The results were compared to the clinically validated tests next generation sequencing and chromosomal microarray test results²² generated in a Clinical Laboratory Improvement Amendments (CLIA) certified laboratory.

### Methods

DNA Extraction from OCT Embedded Frozen Tissue

The study was approved by the Institutional Review of Bord (IRB) of Dartmouth-Hitchcock Medical Center (DHMC, STUDY02001960).

The banked samples were retrieved from the institutional biorepository at the DHMC. Genomic DNA was extracted using the DNeasy Blood & Tissue Kit (Qiagen #69504) with minor modifications for ultrafast extraction. Briefly, 5-10 scrolls of tissue (>5 mm×5 mm) were sectioned at 5 μm thickness onto blank slides in a cryostat machine. A premixed solution containing 180 μl of tissue lysis buffer (buffer ATL) and 2 μl of RNase A (Thermo Scientific #01236994) was added onto the slide and the tissue was scrapped off, then transferred to a 1.5 ml Eppendorf tube. The tube was incubated at 37 °C for 2 minutes, after which 20 μl of protease K was added to the reaction, followed by an additional incubation at 56 °C for 8 minutes. Buffer AL (200 μl) and pure ethanol (200 μl) were then subsequently added to the reactions. The final reaction was mixed thoroughly by vortexing and added to the spin column inserted to the vacuum to facilitate the extraction procedures. Sequential buffer AW1 (500 μl) and AW2 (500 μl) were added to wash the column, which was centrifuged at 20,000×g for 30 seconds for final cleanup. The DNA was eluted with 50-75 μl of AE buffer and its quantity and quality were checked using Nanodrop and Qubit instruments (ThermoFisher).

iSCORED Reaction

Approximately 200-400 ng of input gDNA was used for the iSCORED reaction, followed by bead purification for Nanopore sequencing. The reaction mixture comprised 15 μl in total, which included quick ligase buffer (4.5 μl), quick ligase (1 μl, NEB E6056) and NTAN cocktail mix (1 μl; equal amounts of MseI, BfaI, CviQI and NdeI from NEB). The reaction was incubated at 37 °C for 30 minutes with intermittent cooling/agitation to enhance ligation. Specifically, the reaction was agitated at 900 rpm on the 18 °C pad at reaction time points of 9-10, 14-15, 19-20 and 24-25 minutes. End repair/dA tailing buffer (1.75 μl) and enzyme mix (0.75 μl, NEB E7546) were added to the mixture, which was then incubated at 20 °C for 5 minutes and 65 °C for 5 minutes. For final ligation to the Nanopore motor proteins, a freshly made premix of ligation buffer (LNB, 4 μl), adaptor protein (AMF, 1.5 μl, ONT LSK110 for R9.4.1 flowcells; LA 1.5 μl ONT LSK114 for R10.4.1 flowcells) and quick ligase (1.5 μl) were added to the reaction solution and incubated for 10 minutes at room temperature (20-22 °C) with intermittent mixing. Finally, 10 μl of ddH₂O and 14 μl of PEG deprived AMPure XP beads (Beckman Coulter A63881) were added for standard magnetic bead purification. Of note, the AMPure XP beads were re-suspended in 2.5 M NaCl to eliminate PEG 8000 in the original solution. The beads were washed twice with long fragment buffer (LFB, 80 μl) before eluting into 12 μl elution buffer. Approximately 30-50 ng of ligated DNA were loaded for flongle flowcells (R9.4.1), 100 ng were used for MinION flowcells (R9.4.1) and 50-75 ng for PromethION flowcells (R10.4.1).

### Flowcell Reuse

The MinION (R9.4.1) and PromethION (R10.4.1) flowcells were washed for sequential runs by using the flowcell wash kit (WSH004-XL). Briefly, 400 μl of flowcell wash mix (398 μl of wash diluent and 2 μl of wash mix) were loaded to the priming port to allow a DNase I reaction for 60 minutes at room temperature. The reaction solution was removed from the waste port and storage buffer (500 μl) were loaded into the priming port before storing at 4 °C for next use. A minimum of 800 active pores by flowcell check were required for a successful run. Following the protocol, a typical MinION flowcell (R9.4.1) could be re-used for 5-7 times.

### Normalization of Intrinsic Regional Variability

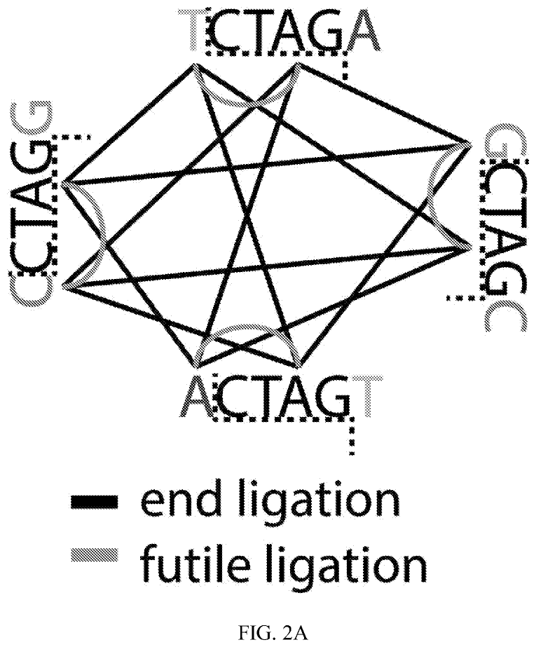

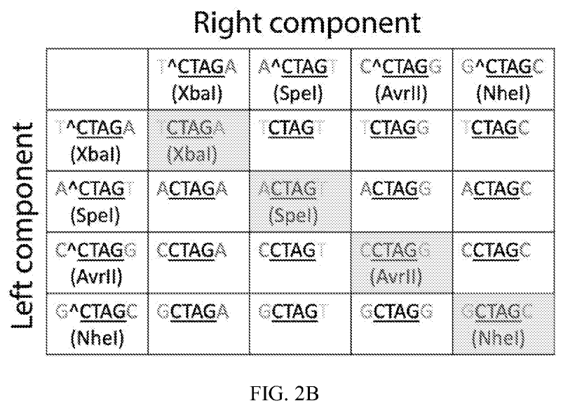

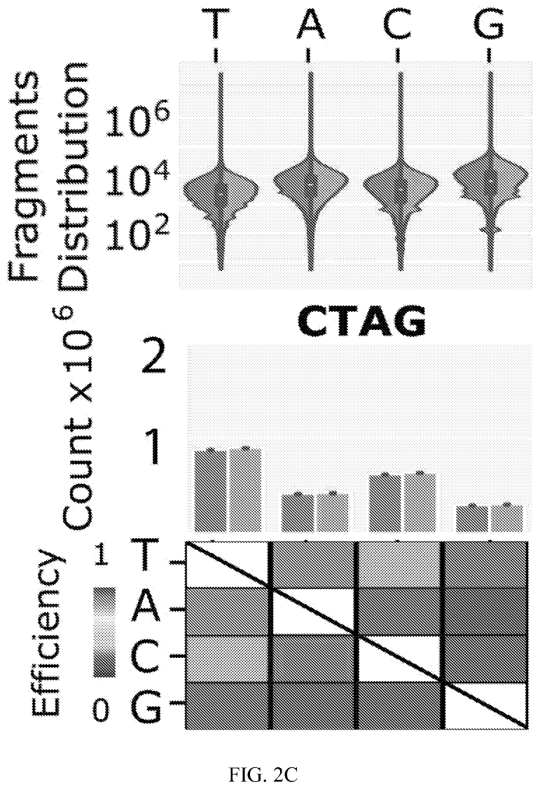

Control human genomic DNA (gDNA; NA12878 from Coriell Institute) was first processed with iSCORED and sequenced extensively (16,564,873 mapped fragments) to establish a reference dataset for normalization to equivalent bins. The bins with counts below 0.2^{th} percentile were removed to eliminate false positive bins as tested in four WT datasets (NA20967, NA12878, NA24385 and NA24631 from Coriell Institute; FIG. 2_S2). An array of the proportion was segmented at the chromosomal level to optimize the resolution of the normalization and increase the sensitivity at which outliers are detected. A normalized vector of ratios (r1), identical in size to the reference array, was created. The distribution of this vector, at the chromosomal level, was employed to detect outliers using three components: i) a threshold of 5 to filter the elements in r1 that were not greater than a specific threshold=5, ii) Z scores to determine the statistical significance of the deviation from the distribution, and iii) the presence of surrounding outlier bins (a minimum of two consecutive bins must be present for a set of datapoints to be considered as outliers).

### Coefficient of Variation Calculation Across Genome

Similar to what was described above, a normalized vector was created using NA12878 as a reference. For this approach, we used three other commonly used control gDNAs (NA20296, NA24385 and NA24631 from Coriell Institute). Each one of these datasets was segmented into independent data subsets (with no overlapping fragments) that vary in the number of mapped fragments. The number of fragments in these datasets were 70k, 200k, 300k, 400k, 500k, 600k, 700k, 800k, 900k, 1M, 1.25M, 1.75M, 2M. For each datasets, the coefficient of variation (CoV) across the genome was calculated to assess the variability. In addition, the behavior of the CoV as the number of fragments change was assessed with the first order derivative of the CoV function.

### Basecalling and Read Filtering

Fast5 files were converted to pod5 with ONT's pod5-file-format (https://github.com/nanoporetech/pod5-file-format) and basecalled with ONT's Dorado v0.2.4 (https://github.com/nanoporetech/dorado) using dna_r9.4.1_e8_fast@v3.4 for r9.4.1 or dna_r10.4.1_e8.2_400bps_sup@v3.5.2 for 10.4.1 with the following setting: --modified-bases 5mCG --emit-moves. The resulting unmapped SAM (uSAM) files were converted to fastq using samtools fastq -TMM, ML to carry the methylation information forward into the fastq header. The resulting FastQs were first processed with Porechop (v0.2.1, https://github.com/rrwick/Porechop) to trim adapter sequences and split reads with internal adapters. Filtered with NanoFilt (2.8.0, [pmid: 29547981]) to remove rare reads greater than 15 kb which represented native genomic reads that did not contribute to our analysis.

Read Processing into Aligned Fragments

The following was adapted from Prabakar³⁶ et al. Briefly, filtered reads were aligned to GRCh37/hg19 using bwa-mem³⁷ (v0.7.17) with the following settings: -x ont2d -k 12 -W 12 -A 4 -B 10 -O 6 -E 3 -T 120. These settings allowed the segmentation of the concatenated reads into individual fragments that were aligned to their respective genomic regions. To ensure accurate quantitative CNV analysis, the duplex reads were identified and excluded from the original uSAM with ONT-Duplex Tools (https://github.com/nanoporetech/duplex-tools). A single member of each pair was then removed from the SAM file using a list of readIDs and Picard (https://github.com/broadinstitute/picard). The genome was then subdivided into either 5,000 or 50,000 genomic bins for CNV and amplification analysis, respectively, and mapped fragments per bin were calculated.

### CNV Analysis

CNV analysis was performed using the Smurf-seq analysis pipeline [pmid: 31287019]. Counts of uniquely mapped fragments to the 5,000 bins in the human genome were normalized for biases in GC content, finally an implementation of DNAcopy³⁸ (v1.74.1) using circular binary segmentation identified breakpoints in bin counts.

### Output Table and Graph

The output table contained a list of at least two consecutive statistically significant outliers (i.e., bins) to minimize the potential of identifying isolated/noisy outliers due to individual genome variation. The table displayed the corresponding position, ratio, Z score at the chromosomal level of each sample, along with commonly amplified gene(s) found in these bins of interest. All annotated genes from the hg19 reference genome were included in the table with 75 commonly amplified genes highlighted in red. The graph was automatically generated if there are significant bins in the sample of interest. Methylation calling and tumor classification

The following were adapted from Patel et al. Rapid-CNS² pipeline³⁴. The filtered reads were aligned to GRCh38/hg38 using bwa-mem³⁷ (v0.7.17) with the following settings: -x ont2d -k 12 -W 12 -A 4 -B 10 -O 6 -E 3 -T 120 -C -Y. Similar to the CNV analysis this broke up the reads into the individual fragments, the -C command allowed the SAM tags for methylation to be moved from the fastq header back into the SAM file. The -Y command turned off soft-clipping which interferes with downstream methylation extraction. The per site methylation is extracted using mbtools (https://github.com/jts/mbtools). A custom python script converted the bedfile to make it compatible with Rapid-CNS². The called methylations were processed using Rapid-CNS² which uses a random forest classifier trained on Illumina BeadChip 450 K methylation array from the Heidelberg reference cohort of brain tumor methylation profiles³⁹.

To determine the minimum time required for methylation classification, we simulated the collection of methylation data over time using samples that had been sequenced for >60 minutes. The data were subdivided into several bins (10, 20, 30, 45, 60 minutes). Sequencing start time was recovered from the uSAM read header and the aligned SAM was filtered accordingly. The data was then processed using our standard analysis pipeline as described above to extract the number of detected methylation features, the methylation classification, and the calibrated scores at each time point.

To study the role of tumor percentage in methylation classification, gDNA from control human frontal lobe was processed with iSCORED. The resulting reads were in silico admixed with datasets from a medulloblastoma, an oligodendroglioma, or a glioblastoma, all of which had >90% tumor percentage and calibrated scores of -0.99 in methylation classification. Reads equivalent to an hour of sequencing on the MinION at different ratios of tumor to control (tumor percentages of: 0-100 in intervals of 10) were used for methylation classification using the Rapid-CNS² pipeline.

### Computer Setting

Our computer system included an Intel® Core™ i9-12900K Processor, 24 cores, 64 GB of RAM, 2Tb of storage and an RTX 3090Ti.

### Results

### Concurrent Fragmentation and Concatenation of DNA Molecules

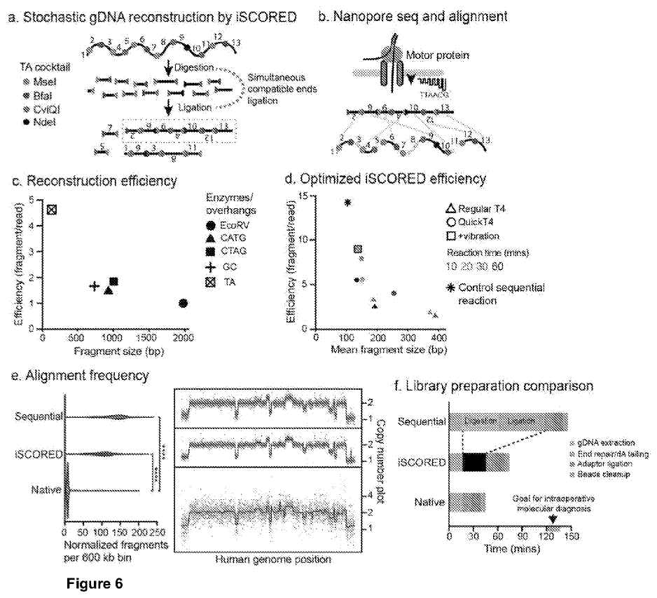

The central concept of iSCORED is simultaneous digestion and ligation of DNA molecules by utilizing a panel of restriction endonucleases (REs) capable of generating compatible cohesive ends. Within the same reaction, DNA ligase catalyzes random re-ligation of the digested fragments (concatemer reconstruction). Irreversible ligation products are generated when cohesive ends are produced by different restriction enzymes. This unidirectional reaction is possible because of the staggered nature of the DNA recognition sequences and actual phosphodiester bond breakage sites (FIG. 6a). Using restriction enzymes capable of generating CTAG overhangs as an example, the digested fragments were concatenated to larger chimeric molecules in the presence of DNA ligase. The likelihood of forming such irreversible ligations increases with the number of different restriction enzymes producing compatible ends.

### Systematic Analysis and Optimization of all Overhang Candidates

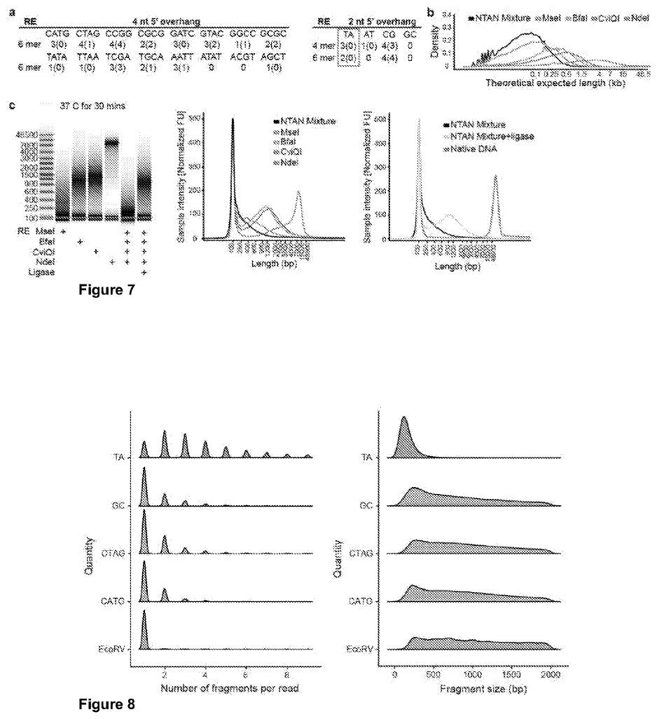

We next examined all existing 4-mer and 6-mer Type IIP REs capable of generating 2-nucleotide and 4-nucleotide overhangs (FIG. 7). Given the palindromic nature of Type IIP REs, there are 16 (=4²) and 4 (=4¹) possible combinations for 4-nucleotide and 2-nucleotide overhangs, respectively. Depending on the RE recognition sequence (4 or 6 bp), the same overhang could be generated by 4¹ or 4² different enzymes; however, some of the theoretical combinations do not exist, and some are partially or completely blocked by DNA methylation (FIG. 7a).

We tested the top four overhang candidates that had the highest number of RE combinations while exhibiting the least possibility of methylation inhibition (FIG. 6c). To quantitatively measure the reconstruction efficiency (i.e., the number of uniquely mapped fragments per sequencing read), we compared combinations generating 4-nt overhangs with those generating 2-nt overhangs. Surprisingly, we found that the efficiency of 4-nt overhang combinations is not superior to that of 2-nt overhang combinations (FIG. 6c, FIG. 8). This is presumably due to the much higher frequency of RE recognition sites. By defining the reconstruction efficiency as numbers of DNA fragments per read, the most efficient combination was the TA overhang cocktail mix that consisted of MseI, BfaI, CviQI, and NdeI, resulting in a mean reconstruction efficiency of 4.6 and mapped fragment of 120 bp. To further optimize the iSCORED reaction, we tested various incubation periods and DNA ligases (FIG. 6d). Our experiments revealed that an incubation period of 30 minutes at 37 °C with intermittent agitation at 18 °C (900 rpm) yielded the highest mean reconstruction efficiency of 8.7 (FIG. 6d). This experimental condition was thus utilized for the remainder of the study.

### Genomewide Aneuploidy Detection in Tumors

To detect large CNV (>10 Mb) and aneuploidy, the sequenced reads were first segmented into individual fragments. These uniquely mapped fragments were then filtered for quality (alignment scores≥120, Methods for details) and assigned to predefined genomic bins (600 kb) for quantitative analysis. High numbers of mapped fragments per bin generated low variability between bins and this helped ensure high confidence in the resulting CNV plot. Finally, circular binary segmentation²³ through DNACopy²⁴ was employed to identify copy number alterations across genomic bins.

The performance of the iSCORED pipeline was compared to the conventional sequential approach (i.e. digestion, purification and ligation) and unprocessed native gDNA. By normalizing the datasets to the same amount of total DNA sequence, the iSCORED exhibited a 16-fold increase in the number of fragments compared to native gDNA sequencing (FIG. 6e). The significant increase in fragment count resulted in low variability, which was critical for detecting copy number changes with high confidence. Specifically, the coefficient of variations (CoV) for the sequential approach, iSCORED and native gDNA sequencing were 0.57, 0.54 and 3.3, respectively. Detection of large CNV and aneuploidy by iSCORED showed 100% concordance with clinically validated chromosomal microarray data (Table 1) and also demonstrated a much higher resolution than short-read based analysis¹⁹ within a comparable timeframe of 2 hours (FIG. 6f, Table 2).

### Refinement of Quantitative Measures to Detect Copy Number Variations

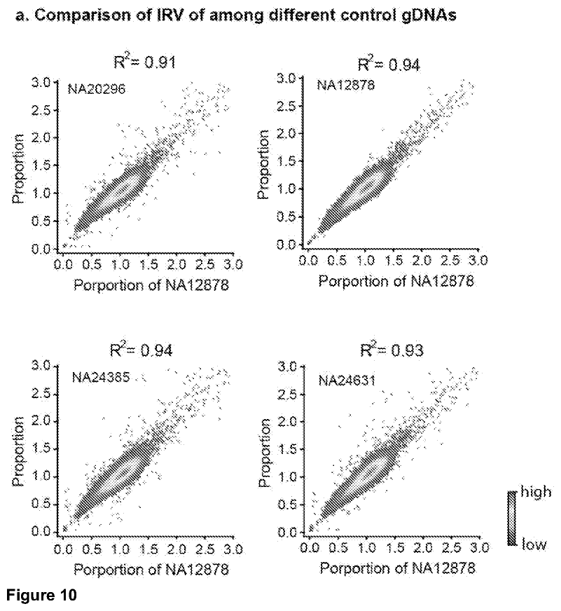

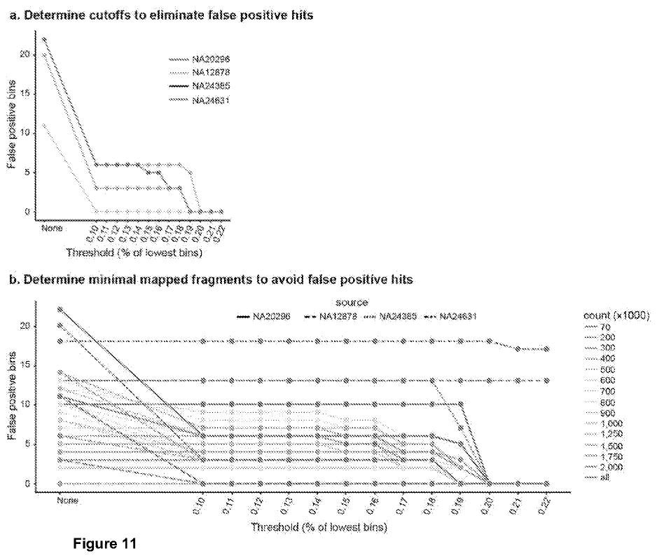

While large bins are effective in detecting aneuploidy, most clinically-relevant gene amplifications occur within a range of hundreds of kilobases to a few megabases^{25,26}. In such cases, using a large bin could result in averaging out the dose change, leading to decreased accuracy of small amplifications. Thus, we refined the bin size to 60 kb, which was similar to the genomic resolution of clinically validated chromosomal microarray analysis (CMA). When using the control human genome (NA12878) to quantify the total mapped fragments in refined 60-kb bins, the numbers mapped fragments fluctuated substantially across the predefined bins (FIG. 7a). Since the iSCORED is a restriction enzyme-based method, this finding was presumably due to variations in the density and distribution of restriction enzymes' cutting sites. We had termed the phenomenon intrinsic regional variability (IRV) (FIG. 10). The background noise might allow for tolerating outliers driven by true underlying copy number changes and affects the detection accuracy. In addition, this fluctuation behavior was inversely related to the amount of the data acquired. Hence, this finding was characterized in the context of the corresponding number of fragments in the genome.

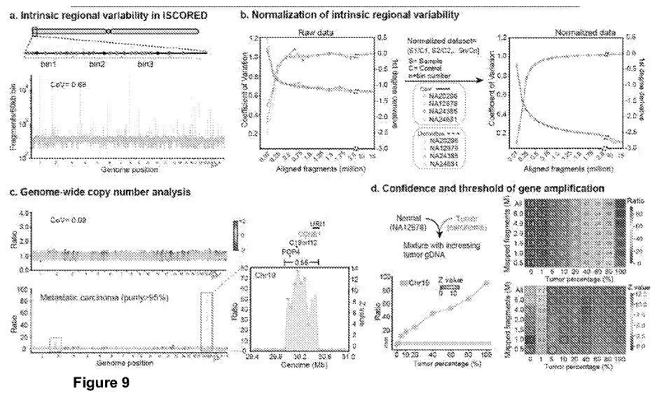

For this purpose, we utilized the coefficient of variation (CoV)²⁷ as a quantitative index, and performed time-lapsed analysis of the sequenced control samples. After comparing the sub-datasets with varying numbers of mapped fragments, we found that significant fluctuations reached a plateau around a CoV of 0.68, even with extensive sequencing (FIG. 9b, left panel). To address this issue, we performed bin-specific normalization by calculating a ratio of the mapped fragments in the sample of interest to those in the commonly used control reference genome (NA12878). This significantly reduced the observed genomic fluctuation by approximately four-fold (FIG. 9b, right panel). The CoV of the normalized data was substantially reduced to 0.09 down from 0.68 in the corresponding non-normalized data.

This design also allowed us to infer the required number of total mapped fragments to reliably identify regions of copy number change. When investigating the slope²⁸ of CoV as a function of the acquired fragments, the inflection point was at a datapoint with mapped fragments of <500k, while the first order derivative function approached a value of zero (0) at about one million mapped fragments (FIG. 9b, right panel). Thus, we determined that acquiring approximately one million mapped fragments was sufficient to reliably detect gene amplification. Bin-specific normalization helped to effectively mitigate the effects of intrinsic regional variability and detect copy number variation.

### Tumor Purity in Detecting Gene Amplification

The iSCORED pipeline was first used to analyze a metastatic adenosquamous carcinoma from the head and neck region (M9 in Table 1). A CCNE1 gene amplification was detected in chromosome 19 (ratio=70, inset in FIG. 9c), accurately identifying a known finding originally detected by next generation sequencing.

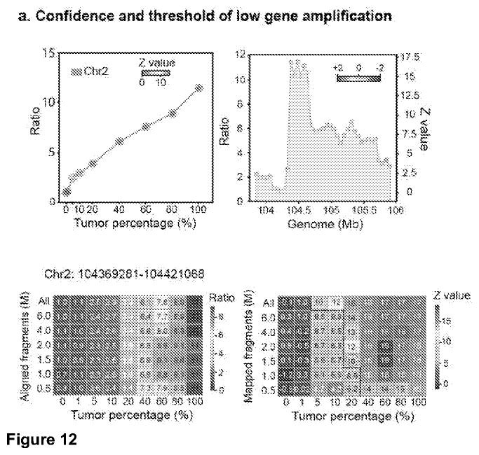

To determine the minimum tumor percentage required for reliably detect CCNE1 amplification, we assessed different compositions of control gDNA mixed with tumor gDNA. This revealed an increasing amplification as tumor percentage increased. To define the accuracy of detection, we implemented a Z-score cutoff of 10. We were able to detect CCNE1 amplification in samples with tumor percentage as low as 5%, using only 500k mapped fragments (FIG. 9d). Additionally, low copy number gain (ratio=11) was also reliably detected with the same parameter, albeit at a higher tumor percentage and with more fragments (20% and 1.5 million fragments, respectively, FIG. 12). Overall, our results demonstrate the effectiveness of the iSCORED pipeline in detecting gene amplifications even in samples with low tumor purity.

### Comprehensive Analysis of the Cohort

We simulated the intraoperative molecular diagnosis by performing blind testing of a cohort of 26 intracranial neoplasms, including 17 primary CNS tumors and 9 metastatic tumors. The performance was timed and the findings were compared to the results from clinically validated next-generation sequencing (TruSight® Tumor 170) and chromosomal microarray analysis (Affymetrix OncoScan)²².

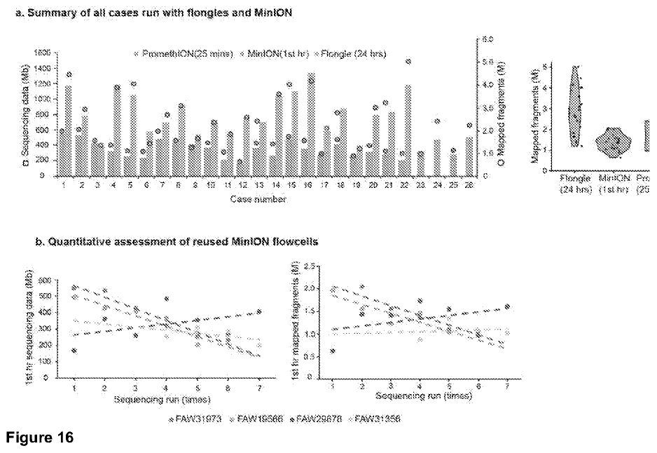

Within one-hour of MinION sequencing, an average 344 Mb (±24 Mb) of data were generated, corresponding to 1.38 million (±0.08×10⁶) mapped fragments (FIG. 16). This is higher than the predetermined required data quantity for confident CNV detection (1×10⁶ mapped fragments, FIG. 9b). Across the 26 investigated samples, the diagnostic accuracy of our approach was 100% in detecting gene amplification of more than 10 copies (95% confidence interval²⁹: 91%-100%, Table 1). One sample was detected to have MYB amplification (ratio of 10.5; case M8) by the iSCORED pipeline, a finding that was not originally uncovered by TST 170 panel but was later verified by a whole exome NGS study (13 copies).

The output genomic graph from the iSCORED pipeline provided precise information on amplified regions and the confidence of detected outliers. EGFR amplification is a molecular defining alterations in glioblastomas. In the six EGFR-amplified glioblastomas we examined, the average amplification regions spanned 1.66±0.44 Mb with an average of 150.5±47 copies. These samples also exhibited varying regions of amplification and doses, which is consistent with the known heterogeneity of glioblastoma^{30,31} (FIG. 18)

### Cost-Effectiveness

To reduce per-test costs, we evaluated the feasibility of re-using flowcells by using the ONT flow cell wash protocol (WSH004) after running each flow cell. Our results indicated that MinION flowcells can be re-used up to 7 times, before it dropped below the minimum requirement of 800 active pores. By employing the wash protocol, our results still demonstrated 100% consistency with independent experiments using new flongle flowcells, indicating the absence of detectable carryover between experiments (FIG. 16). The ability to reuse flow cells resulted in a sequencing cost of $125 per sample (Table 2).

Simultaneous Methylation Classification by iSCORED

Methylation classification of tumor types has emerged as an important diagnostic tool in clinical practice. Brain tumors in particular have benefited from the Heidelberg methylation classifier which classified 91 tumors across 2801 samples³². Given that ONT sequencing can identifying 5-methycytosine (5mC) from native DNA with no additional sample preparation, we extracted methylation information from our sequencing data and classified it with Rapid-CNS², a machine learning-based classifier that has been trained on the Heidelberg dataset.

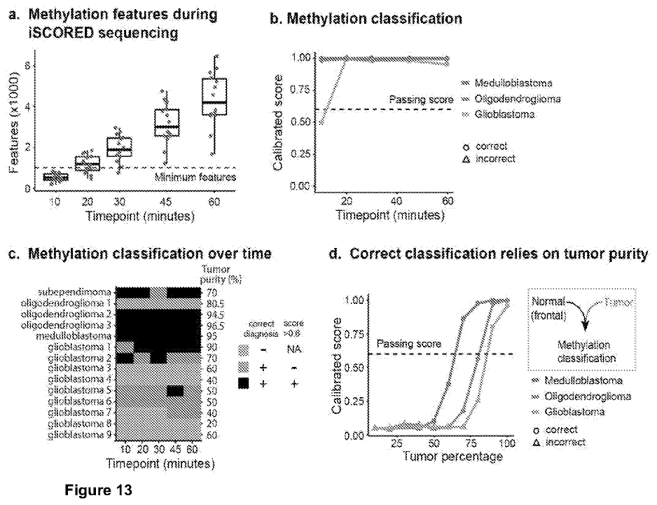

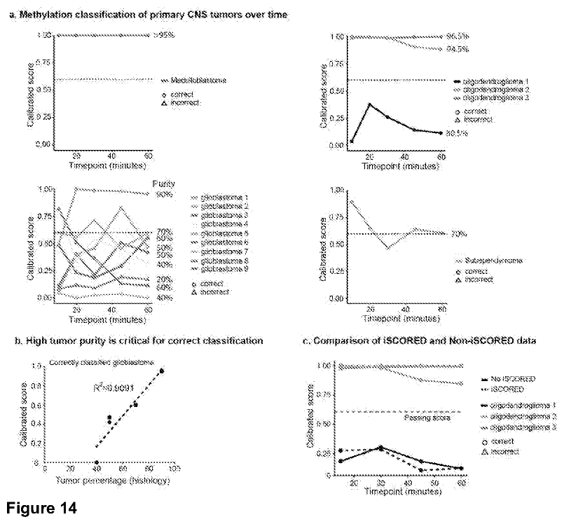

To evaluate the reliability of methylation classification over time, we processed MinION data at five timepoints (10, 20, 30, 45, and 60 minutes) and extracted the number of methylation features that overlapped with the 100k most variable features from the Heidelberg dataset (FIG. 13a). Within 45 minutes, all samples had identified more than 1000 CpG features in all samples, this is the cut-off determined by the original authors³³, and 10 out of 14 samples were correctly classified into tumor subclasses (FIG. 13b and FIG. 14). To investigate if the poor classification scores and misclassification were due to short fragments generated in iSCORED, the results of three oligodendroglioma samples were processed using iSCORED and compared against unprocessed native DNA sequencing results, as in the original publication³⁴. The results revealed comparable classification scores (FIG. 14), indicating that fragmentation in iSCORED did not appear to affect the accuracy of methylation classification.

As the majority of glial neoplasms exhibit an infiltrative growth pattern, resected tumors are often mixed with normal brain parenchyma and inflammatory cells, which could significantly affect the methylation classification accuracy. This is supported by a positive correlation between histologically assessed tumor purity and classification score (FIG. 14). To further assess the impact of tumor purity on classification accuracy, we in silico admixed data from three tumors with the highest classification scores with control CNS tissue (frontal cortex, FIG. 8d). Our analysis revealed a rapid drop in classification scores as tumor purity decreased. Specifically, when the tumor purity was 60%, all classification scores fell below the commonly accepted classification threshold (0.6). Furthermore, when the tumor purity was below 40%, the samples were consistently assigned to incorrect classes. Notably, the medulloblastoma, which had the highest tumor purity, was least affected by control tissue mixing. Therefore, tumor purity is critical for successful tumor methylation classification.

Timeframe for iSCORED Pipeline

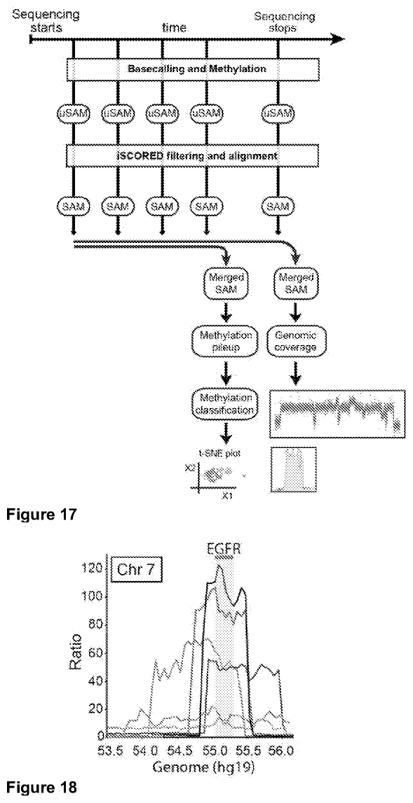

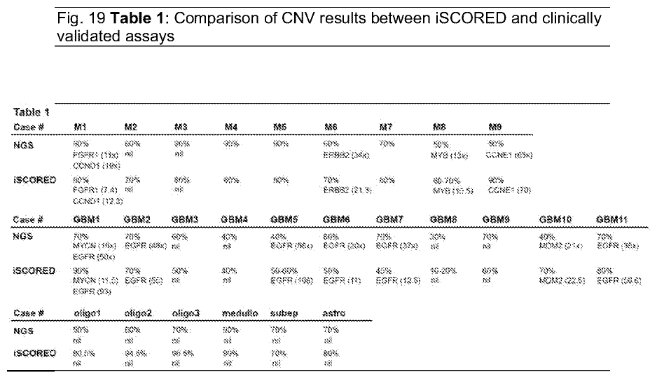

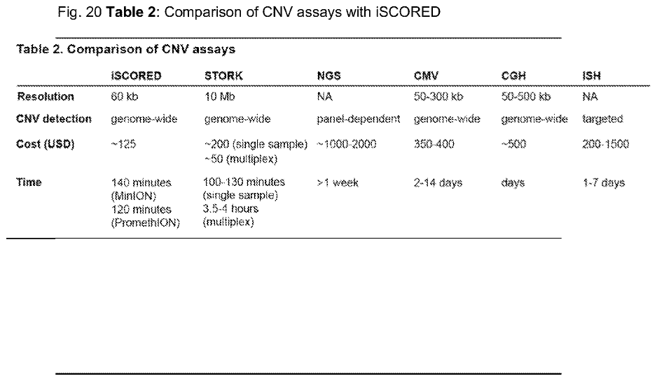

To facilitate intraoperative molecular diagnosis, an analysis pipeline was created that runs in conjunction with sequencing and finishes within minutes of sequencing completion. This pipeline includes a real-time basecalling process along with periodical filtering and alignment of samples for CNV, amplification, and methylation analysis. Once sufficient data is accumulated, the separate files are merged to finalize the analysis (FIG. 17). The pipeline is designed to function on standard computers, eliminating the need for complex and expensive infrastructure (see Methods for specific setup).

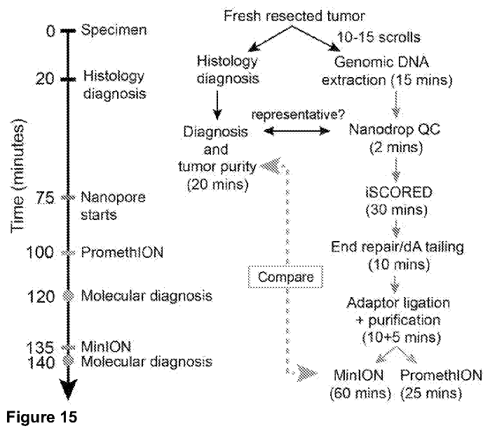

MinION sequencing typically requires 60 minutes to generate sufficient data. With the introduction of P2 Solo (ONT), four of our most recent specimens were analyzed using PromethION technology (R10.4.1). Remarkably, a mere 25 minutes of sequencing yielded 395 Mb (±55 Mb) of data on average, which corresponds to an average of 1.69 million (±37×10⁶) mapped fragments (FIG. 16). Finally, the iSCORED platform and the real-time processing pipeline automatically generate a genome-wide copy number report and methylation classification within 5 minutes and 20 minutes of completing MinION and PromethION sequencing, respectively (140 and 120 minutes after receipt of specimen, FIG. 15). Thus, the iSCORED platform ensures an accurate, fast and inexpensive method for widespread clinical application.

In this study, we present iSCORED, a novel method that can rapidly and affordably generate CNV profiles, detect gene amplifications, and classify tumors by their methylation status. While traditional NGS has had significant impacts in our understanding of the underlying molecular mechanisms of disease, in a clinical setting these technologies, platforms and chemistries are still limited by lengthy turnaround times^{16,17}. Our approach leverages the power of Nanopore sequencing, which is capable of real-time sequencing DNA at speeds of 400 bases per second. While Nanopore has well known capabilities in long-read sequencing, it is not inherently optimized for shorter reads, such as the 120 bp long genomic fragments found in our study during pre-reconstruction. This is primarily due to the potential time wasted on sequencing the adapter regions or awaiting for a new molecule to reload into a pore. We were able to overcome this limitation by concatenating small fragments in the same reaction, significantly improving sequencing efficiency. The iSCORED-based assay produced CNV results that were 100% concordant with clinically validated results in all of our cases. Furthermore, Nanopore sequencing can detect 5-methylcytosine at CpG sites, facilitating tumor methylation classification without any additional sample preparation. Finally, the low cost per sample ($125), ease of infrastructure setup ($6,000-8,000) and unmatched turnaround time (140 minutes) collectively position our method as robust and an invaluable tool for broad-scale clinical applications (Table 2).

The iSCORED platform demonstrates a high accuracy in detecting gene amplifications, with the thresholds set at 5-20% of tumor purities for high and low copy number amplifications, respectively. However, it is important to note that when it comes to methylation classification of primary CNS tumors, sensitivity to tumor purity becomes apparent. Using calibrated score of 0.6 as the cutoff, accurate classification typically requires tumor content exceeding 60%. The phenomenon is not unique to iSCORED platform but also present in other DNA methylation arrays³². Thus, it is crucial to assess the tissue quality and estimate the tumor percentage during the morphology-based intraoperative diagnosis (FIG. 15).

Somatic copy number alterations represent a major type of genetic mutations in cancer initiation, progression and treatment resistance. Among the various cancer types, ovarian carcinoma and sarcoma bear the highest burden of CNVs, accounting for approximately 80% of cases. Following closely are uterine carcinosarcoma and esophageal carcinomas, with approximately 75% presenting CNVs³⁵. These findings underscore the importance of CNV analysis and the applicability of the iSCORED platform in other cancer types. Accurate identification and comprehensive understanding of the prevalence and implications of CNVs in these cancers are pivotal for advancing diagnostic accuracy, prognostic evaluation, and the development of tailored therapeutic interventions. Nanopore sequencing technology is rapidly improving. Currently, the iSCORED genomic resolution is defined and validated at 60 kb, based on the data generated by one hour of MinION sequencing. However, utilizing the recently released P2 Solo device, PromethION flowcells could generate 3-4 times more data in the same timeframe, thereby potentially achieving a 3-4 times higher resolution (15-20 kb per bin). Additional improvements that have been announced by ONT, such as flow cells that can sequence native DNA without adapter ligation, flow cells with 2.5× faster speeds and lower costs. These forthcoming Nanopore sequencing enhancements may potentially enable even more rapid and versatile versions of the iSCORED pipeline.

The contents of all cited references (including literature references, patents, patent applications, and websites) that may be cited throughout this application or listed below are hereby expressly incorporated by reference in their entirety for any purpose into the present disclosure. The disclosure may employ, unless otherwise indicated, conventional techniques of microbiology, molecular biology and cell biology, which are well known in the art.

The disclosed methods may be modified without departing from the scope hereof. It should be noted that the matter contained in the above description or shown in the accompanying drawings should be interpreted as illustrative and not in a limiting sense.

### REFERENCES

The following references are incorporated herein in their entirety:
1. Beroukhim, R. et al. The landscape of somatic copy-number alteration across human cancers. Nature 463, 899-905 (2010).
2. Zack, T. I. et al. Pan-cancer patterns of somatic copy number alteration. Nat. Genet. 45, 1134-1140 (2013).
3. Stratton, M. R., Campbell, P. J. & Futreal, P. A. The cancer genome. Nature 458, 719-724 (2009).
4. Levchenko, A., Kanapin, A., Samsonova, A. & Gainetdinov, R. R. Human Accelerated Regions and Other Human-Specific Sequence Variations in the Context of Evolution and Their Relevance for Brain Development. Genome Biol. Evol. 10, 166-188 (2018).
5. Dumas, L. et al. Gene copy number variation spanning 60 million years of human and primate evolution. Genome Res. 17, 1266-1277 (2007).
6. Marques-Bonet, T. et al. A burst of segmental duplications in the genome of the African great ape ancestor. Nature 457, 877-881 (2009).
7. Heinzen, E. L. et al. Genome-wide scan of copy number variation in late-onset Alzheimer's disease. J. Alzheimers Dis. JAD 19, 69-77 (2010).
8. Lee, W.-P. et al. Copy Number Variation Identification on 3,800 Alzheimer's Disease Whole Genome Sequencing Data from the Alzheimer's Disease Sequencing Project. Front. Genet. 12, 752390 (2021).
9. Hastings, P. J., Lupski, J. R., Rosenberg, S. M. & Ira, G. Mechanisms of change in gene copy number. Nat. Rev. Genet. 10, 551-564 (2009).
10. Pinto, D. et al. Convergence of genes and cellular pathways dysregulated in autism spectrum disorders. Am. J. Hum. Genet. 94, 677-694 (2014).
11. Pinto, D. et al. Functional impact of global rare copy number variation in autism spectrum disorders. Nature 466, 368-372 (2010).
12. Malhotra, D. & Sebat, J. CNVs: harbingers of a rare variant revolution in psychiatric genetics. Cell 148, 1223-1241 (2012).
13. Kallioniemi, A. et al. Comparative genomic hybridization for molecular cytogenetic analysis of solid tumors. Science 258, 818-821 (1992).
14. Wang, D. G. et al. Large-scale identification, mapping, and genotyping of single-nucleotide polymorphisms in the human genome. Science 280, 1077-1082 (1998).
15. Pinkel, D. et al. High resolution analysis of DNA copy number variation using comparative genomic hybridization to microarrays. Nat. Genet. 20, 207-211 (1998).
16. Ciriello, G. et al. Emerging landscape of oncogenic signatures across human cancers. Nat. Genet. 45, 1127-1133 (2013).
17. Malone, E. R., Oliva, M., Sabatini, P. J. B., Stockley, T. L. & Siu, L. L. Molecular profiling for precision cancer therapies. Genome Med. 12, 8 (2020).
18. Gorzynski, J. E. et al. Ultrarapid Nanopore Genome Sequencing in a Critical Care Setting. N. Engl. J. Med. 386, 700-702 (2022).
19. Wei, S. et al. Rapid Nanopore Sequencing-Based Screen for Aneuploidy in Reproductive Care. N. Engl. J. Med. 387, 658-660 (2022).
20. Wang, Z. et al. SMASH, a fragmentation and sequencing method for genomic copy number analysis. Genome Res. 26, 844-851 (2016).
21. Prabakar, R. K., Xu, L., Hicks, J. & Smith, A. D. SMURF-seq: efficient copy number profiling on long-read sequencers. Genome Biol. 20, 134 (2019).
22. Jung, H.-S., Lefferts, J. & Tsongalis, G. Utilization of the oncoscan microarray assay in cancer diagnostics. Appl. Cancer Res. 37, (2017).
23. Olshen, A. B., Venkatraman, E. S., Lucito, R. & Wigler, M. Circular binary segmentation for the analysis of array-based DNA copy number data. Biostat. Oxf. Engl. 5, 557-572 (2004).
24. Seshan, V. E. & Olshen, A. DNAcopy: DNA copy number data analysis.
25. Hung, K. L. et al. Targeted profiling of human extrachromosomal DNA by CRISPR-CATCH. Nat. Genet. 54, 1746-1754 (2022).
26. Helmsauer, K. et al. Enhancer hijacking determines extrachromosomal circular MYCN amplicon architecture in neuroblastoma. Nat. Commun. 11, 5823 (2020).
27. Virtanen, P. et al. SciPy 1.0: fundamental algorithms for scientific computing in Python. Nat. Methods 17, 261-272 (2020).
28. Quarteroni, A., Sacco, R. & Saleri, F. Numerical Mathematics. (Springer New York Springer e-books Imprint: Springer, 2007).
29. Newcombe, R. G. Interval estimation for the difference between independent proportions: comparison of eleven methods. Stat. Med. 17, 873-890 (1998).
30. Parker, N. R., Khong, P., Parkinson, J. F., Howell, V. M. & Wheeler, H. R. Molecular heterogeneity in glioblastoma: potential clinical implications. Front. Oncol. 5, 55 (2015).
31. Louis, D. N. et al. The 2021 WHO Classification of Tumors of the Central Nervous System: a summary. Neuro-Oncol. 23, 1231-1251 (2021).
32. Capper, D. et al. DNA methylation-based classification of central nervous system tumours. Nature 555, 469-474 (2018).
33. Euskirchen, P. et al. Same-day genomic and epigenomic diagnosis of brain tumors using real-time nanopore sequencing. Acta Neuropathol. (Berl.) 134, 691-703 (2017).
34. Patel, A. et al. Rapid-CNS2: rapid comprehensive adaptive nanopore-sequencing of CNS tumors, a proof-of-concept study. Acta Neuropathol. (Berl.) 143, 609-612 (2022).
35. Harbers, L. et al. Somatic Copy Number Alterations in Human Cancers: An Analysis of Publicly Available Data From The Cancer Genome Atlas. Front. Oncol. 11, 700568 (2021).
36. Prabakar, R. K., Xu, L., Hicks, J. & Smith, A. D. SMURF-seq: efficient copy number profiling on long-read sequencers. Genome Biol. 20, 134 (2019).
37. Li, H. & Durbin, R. Fast and accurate short read alignment with Burrows-Wheeler transform. Bioinforma. Oxf. Engl. 25, 1754-1760 (2009).
38. Venkatraman E. Seshan, A. O. DNAcopy. (2017) doi:10.18129/B9.BIOC.DNACOPY.
39. Kuschel, L. P. et al. Robust methylation-based classification of brain tumours using nanopore sequencing. Neuropathol. Appl. Neurobiol. (2022) doi:10.1111/nan.12856.

What is claimed is:

1. A method for constructing a plurality of chimeric DNA molecules, comprising:
a) providing a source DNA molecule; and
b) simultaneously reacting the source DNA molecule with:
i) two or more restriction enzymes; and
ii) a DNA ligase;
to obtain the plurality of chimeric DNA molecules, wherein the two or more restriction enzymes have different restriction recognition sites but produce identical overhangs when cutting a DNA molecule, the two or more restriction enzymes cut the source DNA molecule into smaller DNA fragments, and the DNA ligase ligates the smaller DNA fragments into the plurality of chimeric DNA molecules.

2. The method of claim 1, wherein the two or more restriction enzymes comprise two or more members selected from the group consisting of MseI, BfaI, CviQI, and NdeI.

3. The method of claim 1, wherein the two or more restriction enzymes comprise two or more members selected from the group consisting of XbaI, SpeI, AvrII and NheI.

4. The method of claim 1, wherein the DNA ligase is selected from the group consisting of T3 DNA ligase, T4 DNA ligase, and T7 DNA ligase.

5. The method of claim 1, wherein step (b) comprises two reaction processes (b1) and (b2), wherein in process (b1), the two or more restriction enzymes cut the source DNA molecule into smaller DNA fragments, and in process (b2), the DNA ligase ligates the smaller DNA fragments having complementary overhangs into a larger DNA fragment, wherein the processes (b1) and (b2) are allowed to repeat for N cycles to generate the chimeric DNA molecule, N is an integer greater than 5.

6. The method of claim 5, wherein process (b2) comprises two types of ligations, Type I and Type II ligations, wherein type I ligation results from ligation of two sticky ends generated by same restriction enzyme and can be cut by the same restriction enzyme again, and Type II ligation results from ligation of two sticky ends generated by two different restriction enzymes and cannot be cut by the any of said two different restriction enzyme again after Type II ligation is completed.

7. The method of claim 6, wherein the plurality of chimeric DNA molecules comprises more than 5 smaller DNA fragments ligated together through Type II ligation.

8. The method of claim 1, wherein the smaller DNA fragments have an average length of about 80-100 bp, or about 90 bp (base pairs) when using MseI, BfaI, CviQI and NdeI.

9. The method of claim 1, wherein the reaction of step (b) is completed within 30 minutes or less.

10. The method of claim 1, further comprising a step (c) of sequencing the plurality of chimeric DNA molecules.

11. The method of claim 10, wherein the sequencing is performed by using nanopore sequencing.

12. The method of claim 10, wherein result from the sequencing is analyzed to obtain information selected from the group consisting of copy number variation (CNV), methylation classification, and combination thereof.

13. The method of claim 10, wherein the source DNA molecule is obtained from a mammal.

14. The method of claim 13, wherein the source DNA molecule is obtained from a tumor sample of the mammal.

15. The method of claim 13, wherein the source DNA molecule is obtained during a surgery being performed on the mammal.

16. The method of claim 15, wherein steps (a)-(c) are performed before the surgery is completed and results from steps (a)-(c) are used to guide a surgeon performing the surgery or for implementation of molecularly targeted therapies.

17. A chimeric DNA molecule comprising a plurality of DNA fragments produced by cutting a source DNA fragment with two or more restriction enzymes, wherein the plurality of DNA fragments is stochastically ligated together to form the chimeric DNA molecule, and wherein the two or more restriction enzymes have different restriction recognition sites but produce identical overhangs when cutting a DNA molecule, wherein said chimeric DNA molecule are not digestible by said two or more restriction enzymes.

18. The chimeric DNA molecule of claim 17, wherein the chimeric DNA molecule comprises more than 5 of the plurality of DNA fragments.

19. The chimeric DNA molecule of claim 17, wherein an average length of the plurality of fragments is between 80-100 bp when using MseI, BfaI, CviQI and NdeI.

20. The chimeric DNA molecule of claim 17, wherein an average length of the plurality of fragments is 90 bp when using MseI, BfaI, CviQI and NdeI.

21. A method for reconstructing DNA for single-cell genomic analysis, comprising:
a) lysing single isolated cells to expose genomic DNA in the cells;
b) simultaneously reacting the genomic DNA with:
i) two or more restriction enzymes, wherein the two or more restriction enzymes have different restriction recognition sites but produce identical overhangs when cutting a DNA molecule, wherein the two or more restriction enzymes cut the source DNA molecule into smaller DNA fragments;
ii) short barcodes, wherein the barcodes are unique DNA sequences comprising compatible overhangs to the overhangs produced by the two or more restriction enzymes; and
iii) a DNA ligase, wherein the DNA ligase ligates the smaller DNA fragments and barcodes into a chimeric DNA molecule;
c) amplifying the chimeric DNA molecule; and
d) sequencing the chimeric DNA molecule.

22. The method of claim 21 wherein the single cells are prepared by a process comprising
a) dissociating and suspending a cell population;
b) diluting the cell population to form a cell solution, and
c) transferring the cell solution to a container where single cells are isolated.

23. A kit for constructing a plurality of chimeric DNA molecules, comprising:
i) two or more restriction enzymes;
ii) a DNA ligase; and
iii) an instruction to react a source DNA simultaneously with the two or more restriction enzymes and the DNA ligase to generate the plurality of chimeric DNA molecules,
wherein the two or more restriction enzymes have different restriction recognition sites but produce identical overhangs when cutting a DNA molecule, wherein the two or more restriction enzymes cut the source DNA molecule into smaller DNA fragments, and the DNA ligase ligates the smaller DNA fragments into the plurality of chimeric DNA molecules.

24. The kit of claim 23, wherein the two or more restriction enzymes comprise two or more members selected from the group consisting of MseI, BfaI, CviQI, and NdeI.

25. The method of claim 23, wherein the two or more restriction enzymes comprise two or more members selected from the group consisting of XbaI, SpeI, AvrII and NheI.

26. The method of claim 23, wherein the DNA ligase is selected from the group consisting of T3 DNA ligase, T4 DNA ligase, and T7 DNA ligase.
